(19) United States
(12) Patent Application Publication
(10) Pub. No.: US 2018/0310970 A1
(43) Pub. Date: Nov. 1, 2018

(54) Spinal Implant with Screw Retention and Removal Mechanisms

(71) Applicants: Nicholas Michael Cordaro, Vista, CA (US); Robert Compton, Bixby, OK (US)

(72) Inventors: Nicholas Michael Cordaro, Vista, CA (US); Robert Compton, Bixby, OK (US)

(73) Assignee: Additive Innovations, LLC, Carlsbad, CA (US)

(21) Appl. No.: 15/498,414

(22) Filed: Apr. 26, 2017

(51) Int. Cl. A61B 17/80; A61B 17/86

(52) U.S. Cl. CPC A61B 17/8057; A61B 2017/00946; A61B 17/86

(57) ABSTRACT

The presented device is an orthopedic spinal cage that is inserted from an anteriorly aspect into a patient's intervertebral disc space. The device includes a cage to maintain vertebral separation and allow for fusion. Threaded screws allow for a matting and lag feature to prevent screw back out, provide a tactile event once fully inserted, and then to provide a stepped feature for reliable screw removal with minimal axial force. These features may be adapted to any orthopedic or other application requiring the thread screw features.

### CROSS REFERENCE

Provisional Application No.: 62/327,950

Provisional Application Filling Date: 26 Apr. 2016

Confirmation No. 9955

### STATEMENT OF FEDERALLY SPONSORED RESEARCH OR DEVELOPMENT

This application and device have received no federally sponsored research or development assistance.

### BACKGROUND

U.S. Classification A61F2/44, Joints for the spine, e.g. vertebrae, spinal discs

The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1β and TNF-α as well as matrix metalloproteinases (“MMPs”). These cytokines and MMPs help regulate the metabolism of the nucleus pulposus cells.

In some instances of disc degeneration disease (DDD), gradual degeneration of the intervertebral disc is caused by mechanical instabilities in other portions of the spine. In these instances, increased loads and pressures on the nucleus pulposus cause the cells within the disc (or invading macrophages) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

One proposed method of managing these problems is to remove the problematic disc and replace it with a porous device that restores disc height and allows for bone growth therethrough for the fusion of the adjacent vertebrae. These devices are commonly called “fusion devices” or “fusion cages”.

Current interbody fusion techniques typically include not only an interbody fusion cage, but also supplemental fixation hardware such as fixation screws. This hardware adds to the time, cost, and complexity of the procedure. It also can result in tissue irritation when the cage's profile extends out of the disc space, thereby causing dysphonia/dysphagia in the cervical spine and vessel erosion in the lumbar spine. In addition, the fixation hardware typically includes a secondary locking feature, which adds to the bulkiness of the implant and time required for the procedure. Furthermore, existing fixation hardware may prevent the implantation of additional hardware at an adjacent location, and so require removal and potentially extensive revision of a previous procedure.

US Published Patent Application 2008-0312698 (Bergeron) discloses a device and system for stabilizing movement between two or more vertebral bodies and methods for implanting. Specifically, the embodiments provide medical professionals with the ability to selectively position and orient anchors in bony tissue and then attach a plate to the pre-positioned anchors. The plate assembly, once positioned on the anchors, prevents the anchors from backing out of the bony tissue. Furthermore, in situations in which it is desirable to provide spacing between two vertebral bodies, a spacer may be fixedly connected to the plates for positioning between two vertebral bodies. The spacer may further function as a lock out mechanism, or may be rotatably connected to the plates to maintain rotational freedom. The spacer may incorporate connection features or attachment features.

U.S. Pat. No. 4,904,261 (Dove) discloses a spinal implant, e.g., to replace an excised disc, comprising a rigid generally horseshoe shape of biocompatible material, such as carbon-fibre reinforced plastics, having upper and lower planar faces converging towards the ends of the horseshoe, and at least one hole from each planar face emerging in the outer curved face of the horseshoe, to enable the horseshoe to be fixed by screws inserted through one or more selected holes in each plurality from the ends in the outer curved face into respective adjacent vertebrae, with the screw heads bearing against shoulders, and with the space bounded by the inner curved face of the horseshoe available for the insertion of bone graft or a bone graft substitute.

U.S. Pat. No. 6,579,290 (Hardcastle) discloses a surgical implant for fusing adjacent vertebrae together comprising a body portion with spaced arms. The body portion has passages to receive surgical fixing screws engaged in holes drilled in the vertebrae for securing the body portion to the anterior faces of the vertebrae to be fused. The arms extend into a prepared space between the vertebrae to be fused. Graft material is packed between the arms. Each surgical fixing screw has an externally screw-threaded shank divided into wings which can be outwardly deformed to anchor the shank in the hole. Each surgical fixing screw also has a head which can be transformed between a laterally expanded condition and a laterally contracted condition to permit the head to be interlocked with the implant

U.S. Pat. No. 6,342,074 (Simpson) discloses a spinal fusion implant and method for maintaining proper lumbar spine curvature and intervertebral disc spacing where a degenerative disc has been removed. The one-piece implant comprises a hollow body having an access passage for insertion of bone graft material into the intervertebral space after the implant has been affixed to adjacent vertebrae. The implant provides a pair of screw-receiving passages that are oppositely inclined relative to a central plane. In one embodiment, the screw-receiving passages enable the head of an orthopedic screw to be retained entirely within the access passage. A spinalfusion implant embodied in the present invention may be inserted anteriorly or laterally.

U.S. Pat. No. 6,972,019 (Michelson) discloses a spinal fusion implant for insertion between adjacent vertebral bodies that has opposed upper and lower surfaces adapted to contact each of the adjacent vertebral bodies from within the disc space, a leading end for insertion between the adjacent vertebral bodies, and a trailing end opposite the leading end. The trailing end has an exterior surface and an outer perimeter with an upper edge and a lower edge adapted to be oriented toward the adjacent vertebral bodies, respectively, and a plurality of bone screw receiving holes. At least one of the bone screw receiving holes is adapted to only partially circumferentially surround a trailing end of a bone screw received therein. At least one of the bone screw receiving holes passes through the exterior surface and one of the edges so as to permit the trailing end of the bone screw to protrude beyond one of the edges.

US Patent Publication 2009-0030520 (Biedermann) discloses a fixation device for bones that includes a member which is to be fixed to one or more bones and has at least one bore for receiving a bone screw, wherein the at least one bore comprises a first internal thread portion. The bone screw has a first shaft section provided with a first external thread portion arranged to cooperate with the internal thread portion of the at least one bore, and a head section having a diameter larger than that of the shaft section to provide a catch arranged to engage with a stop formed in the bore. The bone screw further has a second shaft section which includes a clearance groove extending between the catch of the head section and the external thread of the first shaft section. The clearance groove allows disengagement of the two thread portions, such that the bone screw is prevented from being unscrewed off the bore when it is loosened within the adjacent bone. The member can also include a side wall of a cage used in an intervertebral implant device, or can represent a plate of a bone plate assembly.

### BRIEF SUMMARY OF THE INVENTION

The presented invention is an orthopedic spinal cage that is inserted from an anteriorly aspect into a patient's intervertebral disc space. The device includes a cage to maintain vertebral separation and allow for fusion. Threaded screws allow for a matting and lag feature to prevent screw back out, provide a tactile event once inserted, and then to provide a stepped feature for reliable screw removal with minimal axial force. These features may be adapted to any orthopedic or other application requiring the thread screw features.

### BRIEF DESCRIPTION OF THE DRAWINGS

FIGS. 1A-1C are side and cross-sectional views of the device within common vertebra bodies with the bone screw partially engaged within the cage.

FIGS. 2A-2C are side and cross-sectional views of the device within common vertebra bodies with the bone screw nearing full engagement into the cage.

FIGS. 3A-3C are side and cross-sectional views of the device within common vertebra bodies with the bone screw past the tactical engagement of the cage.

FIGS. 4A-4C are side and cross-sectional views of the device within common vertebra bodies with the bone screw fully engagement into the cage with the screw free to rotate or lag the vertebral body as needed.

FIGS. 5A-5C are side and cross-sectional views of the device within common vertebra bodies with the bone screw fully engagement into the cage with the screw free to rotate or lag the vertebral body as needed with noted diametric references.

FIGS. 6A-6C are side and cross-sectional views of the device within common vertebra bodies with the bone screw partially engaged within the cage with noted diametric references.

FIGS. 7A-7C are side and cross-sectional views of the cage device within common vertebra bodies showing the treading of the material.

### DETAILED DESCRIPTION OF THE INVENTION

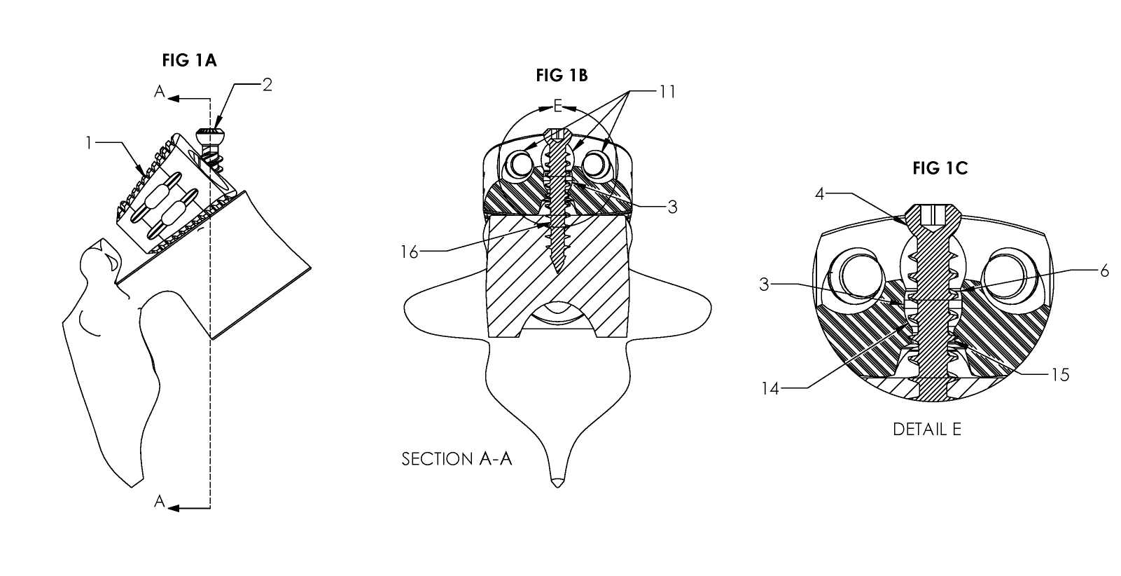

Referring to FIG. 1A, the interbody implant consists of a polymer spacer body 1 and a plurality of bone screws 2 used to secure the polymer implant 1 to the surrounding vertebral bodies 13.

Referring to FIGS. 1B & C apertures 11 in the polymer implant 1 are fabricated with a cavity 3 designed to allow passage of the threaded portion 16 of the bone screw 1 and to capture and accommodate the hemispherical shaped head 4 of the bone screw 2.

One opening of the cavity 3 has an annular feature 6 that is smaller in diameter than the hemispherical diameter of the bone screw 4 head. An opposing opening in the cavity 3 has a spherical shaped surface 14 and reduced diameter passage 15.

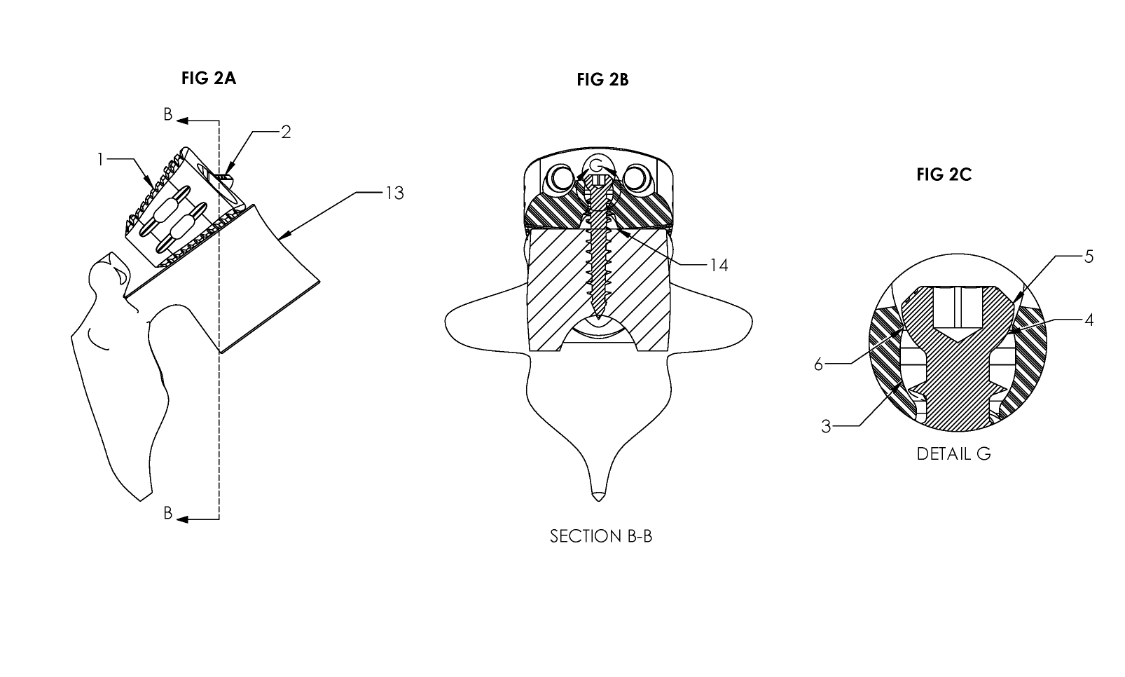

Referring to FIGS. 2A, B & C, as the bone screw 2 is being threaded into the vertebral end plate 14, its hemispherical head 4 will come in contact with the smaller diameter region 6 of the implant bone screw aperture 11. Referring to FIGS. 3A, B & C, during the bone screw 2 placement portion of the procedure, the largest diameter region 5 of the bone screw head 4 will be driven through the smaller diameter annular feature 6, in the polymer implant 1. The smaller diameter annular feature 6 will reduce any tendency to back out of the vertebral end plate 14 the bone screw 2 may exhibit.

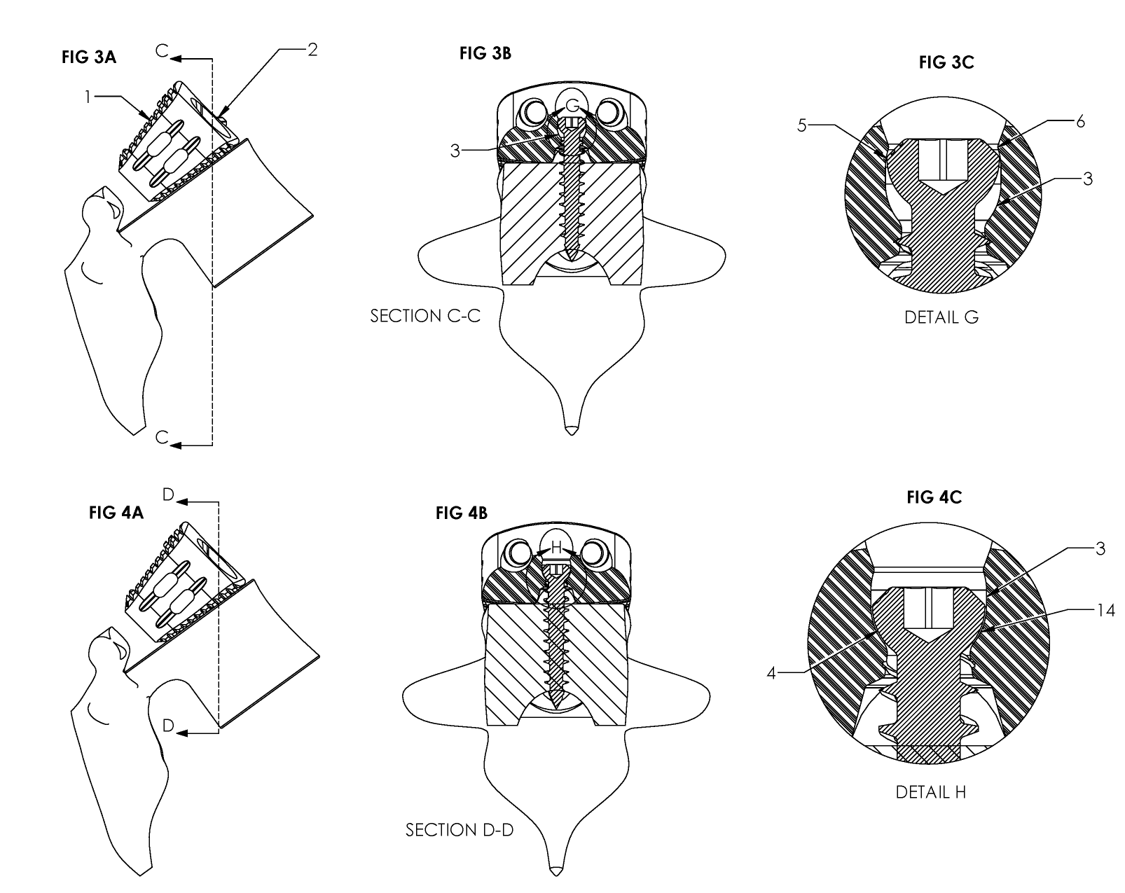

FIG. 4C shows the hemispherical head of the bone screw 4 completely seated in a mating spherical pocket 14 the implant 1. The practitioner will receive both tactile and audible feedback during bone screw 2 placement indicating the bone screw 2 is nearly completely seated in the implant 1 as shown in FIG. 4C.

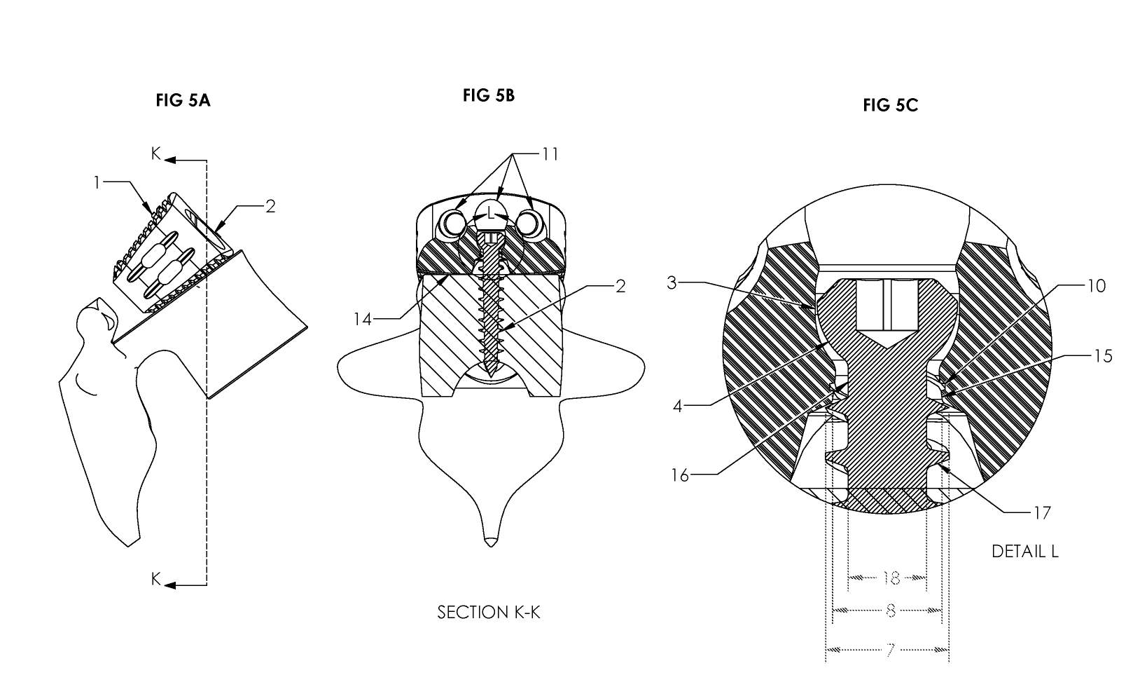

FIGS. 5A, B & C illustrates additional features of the polymer implant 1 and bone screw 2 intended to reduce any tendency to back out of the vertebral end plate 14 the bone screw 2 may exhibit. A region 15 at one end of the cavity 3 formed in apertures 11 in the polymer implant 1 has a diameter 8 less than the major diameter 7 of the bone screw 2. Threads 17 on the bone screw 2 are relieved to the minor diameter 18 of the thread form near the head 4 of the bone screw 2. Any tendency of the bone screw to back out of the vertebral end plate 14 will be resisted as the non-relieved portion of thread on the bone screw 2 encounters the reduced diameter portion 15 of the implant aperture 11.

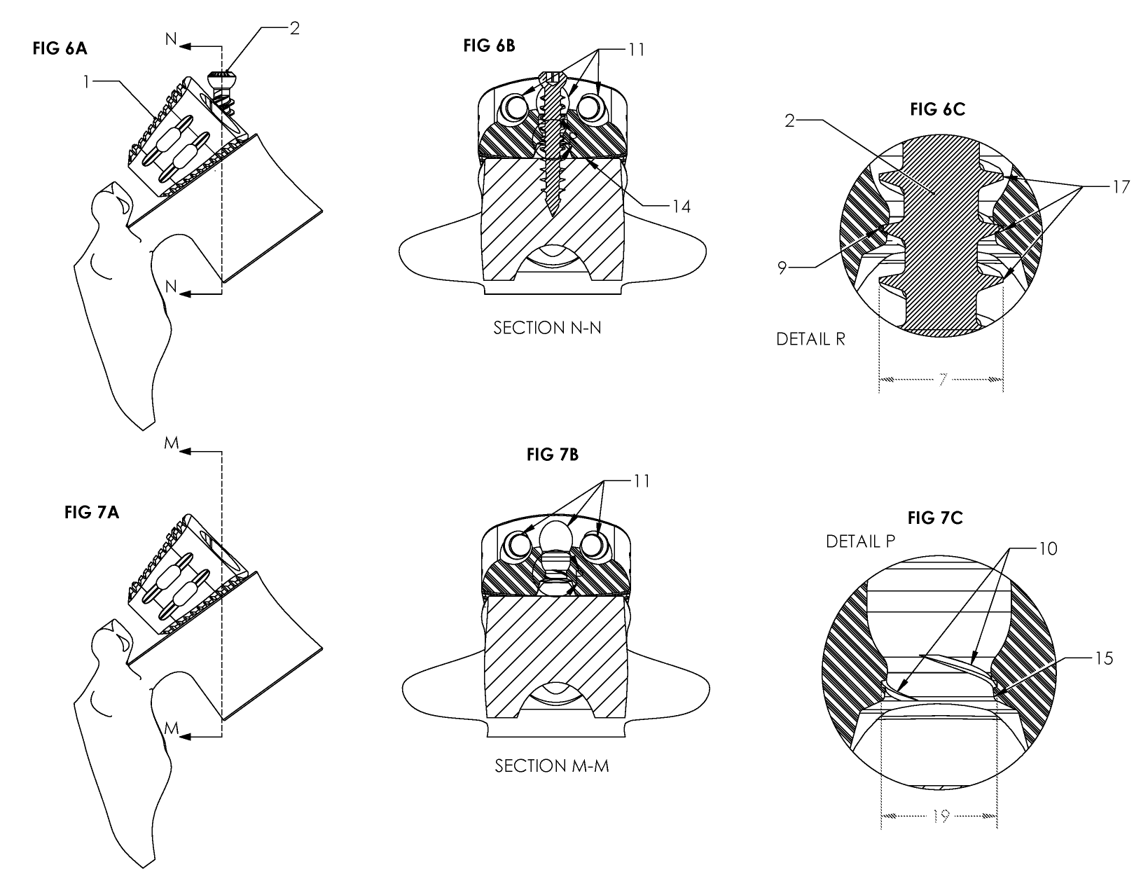

Bone screw 2 placement will create grooves 10 in the reduced diameter region 15 of the implant aperture 11 wall as the polymer material will plastically deforms to accommodate passage of the bone screw 2 threads 17. Significant elastic deformation of the polymer material will also occur allowing the grooves 10 to recover to a diameter 19 less than that of the major diameter 7 of the bone screw. This material interference 9 is illustrated in FIG. 6C and will reduce any tendency to back out of the vertebral end plate 14 the bone screw 2 may exhibit.

Flat section X allows, during screw removal, the threads to first engage under axial torque prior to the hemispherical head 4 coming into contact with the smaller diameter region 6. This will allow the applied axial torque to be converted to linear motion from the created grooves 10 in the reduced diameter region 15 of the implant aperture 11 in corporation with the bone screw 2 threads 17.

What is claimed is:

1. A fixation device for bones, comprising:
a member configured to be fixed to one or more bones and has at least one bore for receiving a bone screw for insertion through at least one bore in the bone, wherein the at least one bore comprises a first internal cavity having an annular feature, annular cavity, and a reduced diameter passage;
the bone screw having:
a first shaft section provided with a first external thread portion configured to thread into the reduced diameter passage portion of the at least one bore;
a head section having a diameter larger than reduced diameter passage portion of the at least one bore to provide a catch configured and where the head section is larger than annular feature of the at least one bore to provided restricted passage; and
a second shaft section comprising a clearance groove extending between the diameter of the head section and the external thread of the first shaft section
wherein the member allows passage of the threaded portion of the bone screw by means of tapping into the reduced diameter passage and to capture and accommodate the hemispherical shaped head of the bone screw.

2. The fixation device according to claim 1 wherein the annual feature has a least a partially smaller radius on at least one side of the internal cavity wherein it deforms to allow the bone screw head to pass by with a tactile feel.

3. The fixation device according to claim 2 wherein a fully introduced bone screw will first engage the member reduced diameter passage with the bone screw first external thread portion before the bone screw head section contacts the member annular feature during screw removal.

4. The fixation device according to claim 3 where the member is a polymer and the bone screw is a metallic device.
